(19) United States
(12) Patent Application Publication
(10) Pub. No.: US 2022/0211638 A1
(43) Pub. Date: Jul. 7, 2022

(54) PRODUCTS OF MANUFACTURE AND METHODS FOR TRANSDERMAL DELIVERY OF PHARMACEUTICALS, ELECTROLYTES, AND NUTRICEUTICALS

(71) Applicant: UNIVERSITY OF NORTH TEXAS, Denton, TX (US)

(72) Inventors: Guido VERBECK, Lewisville, TX (US); R. Scott MCKINLEY, West Vancouver (CA)

(21) Appl. No.: 17/606,718

(22) PCT Filed: May 28, 2020
(86) PCT No.: PCT/US2020/034885

§ 371 Date: Oct. 26, 2021

Related U.S. Application Data
(60) Provisional application No. 62/853,453, filed on May 28, 2019.

(51) Int. Cl. A61K 9/70; A61K 45/06

(52) U.S. Cl. CPC A61K 9/7084; A61K 45/06

(57) ABSTRACT

In alternative embodiments, provided are products of manufacture and methods for using them, for the transdermal or transmucosal delivery of payloads and active agents such as pharmaceuticals, electrolytes, natural products and nutraceuticals. In alternative embodiments, provided are products of manufacture that utilize a nanoporous substrate coupled with controlled melt or solubilization of polymers for the delivery of the payloads and active agents or electrolytes transdermally.

### RELATED APPLICATIONS

This Patent Convention Treaty (PCT) International Application claims benefit of priority of U.S. Provisional Application Ser. No. 62/853,453 filed May 28, 2019. The aforementioned application is expressly incorporated herein by reference in its entirety and for all purposes.

### TECHNICAL FIELD

This invention generally relates to medical devices and drug delivery. In alternative embodiments, provided are products of manufacture and methods for using them, for the transdermal or transmucosal delivery of compounds and active agents such as pharmaceuticals, electrolytes, natural products and nutraceuticals. In alternative embodiments, provided are products of manufacture that utilize a nanoporous substrate coupled with controlled melt or solubilization of polymers for the delivery of the compounds, active agents or electrolytes transdermally.

### BACKGROUND

The Iran-Iraq war and the Tokyo sarin attack provided valuable lessons in treatment of chemical warfare attacks¹. During these attacks first responders subjected to the chemical warfare agent (CWA) were treated using the atropine “buddy system”, which allows for military personnel and first responders to carry three 2 mg atropine injections and dose themselves and others every 5-10 minutes or until the symptoms dissipate². Unfortunately, this requires first responders to recognize the symptoms, provide accurate dosages, and monitor the symptoms of each person that was injected. This lack of automation in CWA attacks reduces the time the first responder can focus on evacuating the area or searching for individuals to extract from the contaminated zone. Clearly, there is a need for better means to quickly deliver chemical reagents.

### SUMMARY

In alternative embodiments, provided are products of manufacture for the transdermal or transmucosal delivery of compounds, comprising at least three layers:

(a) an inner layer designed to be approximate to a mucous membrane or skin comprising a heat sensitive porous material, wherein the heat sensitive porous material becomes porous, semi-solid or soluble at a temperature of between about 50° C. to 68° C., between about 55° C. to 65° C., or at about 60° C., or the heat sensitive porous material comprises a plurality of pores which expand in pore size or pore diameter at a temperature of between about 50° C. to 68° C., between about 55° C. to 65° C., or at about 60° C., or the viscosity of the heat sensitive porous material increases at a temperature of between about 50° C. to 68° C., between about 55° C. to 65° C., or at about 60° C.,

wherein optionally the heat sensitive porous material comprises a gelatin film or a hydrogel, or equivalents, and optionally the gelatin film comprises a fish gelatin or equivalent, and optionally the fish gelatin comprises a fish skin gelatin, a bovine gelatin, a porcine gelatin or a hydrogel or equivalents,

and optionally the heat-sensitive porous material or equivalent is layered on or embedded in a microporous polyolefin silica substrate or equivalent,

and optionally the average pore size or pore diameter increases to between about 50 nm to about 50 μm when the temperature of the heat sensitive porous material increases to between about 30° C. to about 68° C.,

and optionally the viscosity of the heat sensitive porous material increases by between about 5% to about 100% when the temperature of the heat sensitive porous material increases to between about 50° C. to 68° C.;

(b) an inner (or middle) layer comprising a plurality of nanotransferosomes, lipid bodies, nano-liposomes, liposomes, nano-liposomes or equivalent particles or equivalents or combinations thereof comprising or having contained therein a payload or an active agent,

wherein optionally the payload or active agent comprises a small molecule, a protein or peptide, a polysaccharide, a lipid, an ion, a reagent, a pharmaceutical or drug, an electrolyte, a natural product and/or a nutraceutical,

and optionally all or substantially all of the plurality of payload-comprising nanotransferosomes, lipid bodies, nano-liposomes, liposomes, nano-liposomes or equivalent particles have a diameter of less than about 30 nm, or at or less than about 25 nm, or optionally all or substantially all of the plurality of payload-comprising nanotransferosomes, lipid bodies, nano-liposomes, liposomes, nano-liposomes or equivalent particles have a diameter averaging between about 20 and 30 nm, or between about 20 and 25 nm,

and optionally the plurality of nanotransferosomes, lipid bodies, nano-liposomes, liposomes, nano-liposomes or equivalent particles are in the inner (or middle) layer in or at a concentration of between about 1 mg/mL and 1000 mg/mL, or between about 5 mg/mL and 100 mg/mL, or about 10 mg/mL,

and optionally the plurality of payload-comprising nanotransferosomes, lipid bodies, nano-liposomes, liposomes, nano-liposomes or equivalent particles are embedded in a microporous, mesoporous or nanoporous polyolefin silica substrate, or equivalent, or are embedded in an organic or inorganic porous framework; and

(c) an outer layer comprising or substantially comprising a Positive Temperature Coefficient (PTC) material,

and optionally the PTC material comprises a poly-crystalline ceramic or silica material, or a microporous polyolefin silica substrate,

and optionally the poly-crystalline ceramic or silica material, or the microporous polyolefin silica substrate, comprises pores, wherein all or substantially all of the pores are less than about 30 nm in diameter, or less than about 25 nm, or average less than about 30 nm in diameter,

and optionally the poly-crystalline ceramic material or the microporous polyolefin silica substrate comprises a dopant to make the poly-crystalline ceramic material or the microporous polyolefin silica substrate conductive,

wherein optionally the PTC material is printed onto, attached to or embedded on or contained within a polymer, a textile, a fabric or a cloth,

wherein optionally the polymer comprises a polyester, a polyethylene terephthalate (PET), an acrylic-comprising polymer or equivalent.

In alternative embodiments of products of manufacture as provided herein:
the products of manufacture further comprise a fourth layer comprising a micro-porous or a mesoporous substrate comprising an embedded, or having contained therein, a gas, a gel or a liquid which expands when heated to between about 50° C. to 68° C., wherein optionally the embedded or contained gas, gel or liquid expands in volume by about 10% to 100%;
the products of manufacture are fabricated on or as a wearable product or a device, wherein the wearable product or device is or comprises a band, an eyeglass, a watch, clothes, shoes, a hat, a prosthesis, a wound or burn dressing, a patch, or any garment or wearable product;
the wearable product or device comprises an adhesive such that the product of wearable product or device can be removably attached or adhered to the skin or a mucous membrane;
the products of manufacture further comprise a heating module capable of heating the product of manufacture, the fourth layer comprising a nanoporous, micro-porous or a mesoporous substrate, and/or the heat sensitive porous material of the inner later to a temperature of between about 55° C. to about 65° C., or to about 60° C., wherein optionally the heating module comprises a switch which when manually or electronically activated turns the heating module on (to produce heat) or off, and/or
the product of manufacture further comprise an antenna (optionally an ultra-thin magnetic spiral antenna) or equivalent, and a near-field communication (NFC) chip or equivalent, and the ultra-thin magnetic spiral antenna is capable of receiving a remote electromagnetic signal and transmitting the signal to the near-field communication (NFC) chip, and the NFC chip is operatively connected to the heating module.

In alternative embodiments, provided are methods of transdermally or transmucosally administering a payload or an active agent to an individual in need thereof, comprising:

(a) providing a product of manufacture as provided herein, wherein the nanotransferosomes, lipid bodies, nano-liposomes, liposomes, nano-liposomes or equivalent particles comprise or have contained therein the payload or active agent;

(b) applying, contacting or placing in close approximation the inner surface of the product of manufacture to a skin or a mucous membrane; and

(c) (i) applying sufficient heat for a sufficient time to the heat sensitive porous material to melt or sufficiently solubilize the heat sensitive porous material to allow passage of a plurality of nanotransferosomes, lipid bodies, nano-liposomes, liposomes, nano-liposomes or equivalent particles from (or out of) the product of manufacture to the surface of the skin or the mucous membrane, or

(ii) generating a remote signal that is transmitted to the antenna and the near-field communication (NFC) chip to generate a sufficient signal to activate the heating module for a sufficient time to the heat sensitive porous material to melt or sufficiently solubilize the heat sensitive porous material to allow passage of the plurality of nanotransferosomes, lipid bodies, nano-liposomes, liposomes, nano-liposomes or equivalent particles from (or out of, or diffuse out of) the product of manufacture to the surface of the skin or the mucous membrane,

and optionally sufficient heat for a sufficient time is applied to the heat sensitive porous material such that all, substantially all or only a portion of the active agent-comprising nanotransferosomes, lipid bodies, nano-liposomes, liposomes, nano-liposomes or equivalent particles pass from (or out of) the product of manufacture to the surface of the skin or the mucous membrane.

In alternative embodiments of methods as provided herein, two, three or multiple pulses of sufficient heat for a sufficient time are applied to the product of manufacture such that only portions of the active agent-comprising nano-transferosomes, lipid bodies, nano-liposomes, liposomes, nano-liposomes or equivalent particles pass from (or out of) the product of manufacture to the surface of the skin or the mucous membrane pass from the product of manufacture to the skin or mucous membrane,

and optionally the amount of payload- or active agent-comprising nano-transferosomes, lipid bodies, nano-liposomes, liposomes, nano-liposomes or equivalent particles that pass from (or out of) the product of manufacture to the surface of the skin or the mucous membrane is controlled by the amount of heat generating by the heating module or the amount of time the heating module is heated,

and optionally for each pulse heat between about 5% to about 95%, or 10% to about 90%, or about 20% to 80%, of the payload- or active agent-comprising nanotransferosomes, lipid bodies, nano-liposomes, liposomes, nano-liposomes or equivalent particles pass from the product of manufacture to the skin or mucous membrane.

In alternative embodiments, provided are uses of product of manufactures as provided herein for transdermally or transmucosally administering a payload or an active agent to an individual in need thereof.

In alternative embodiments provided are products of manufacture as provided herein for use in transdermally or transmucosally administering a payload or an active agent to an individual in need thereof.

In alternative embodiments, provided are kits comprising a product of manufacture as provided herein.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

The drawings set forth herein are illustrative of exemplary embodiments provided herein and are not meant to limit the scope of the invention as encompassed by the claims.

FIG. 1 illustrates an exemplary device as provided herein set upon the skin, where the lower two images illustrate how a payload, or nanotransferosomes, lipid bodies, nano-liposomes or liposomes or nano-liposomes loaded with (or comprising) a payload, is released onto, upon, or into the skin upon application of heat to the exemplary device.

FIG. 2 schematically illustrates an exemplary configuration of a device as provided herein for transdermal chemistry delivery on top of a skin layer, FIG. 4 showing payload (such as, e.g., small molecules, nanotransferosomes, nanoparticles, lipid bodies, liposomes, electrolytes, pharmaceuticals, nutraceuticals and the like) (or wherein the nanotransferosomes, lipid bodies, nano-liposomes or liposomes or nano-liposomes themselves are loaded with small molecules, nanoparticles, electrolytes, drugs, pharmaceutical and the like) and an exemplary gelatin or gelatin-like material melt (where the term “gelatin-like material” comprises hydrogels, natural or synthetic gelatins, cellular barriers, agarose, alginate, lipid bilayers, polysaccharides) embedded in a porous material, over which is a PTC heating control, over which is a patch controller.

FIG. 3 schematically illustrates an exemplary configuration of a device as provided herein for transdermal chemistry delivery on top of a skin layer, the figure showing a exemplary gelatin or gelatin-like material melt (where the term “gelatin-like material” comprises hydrogels, natural or synthetic gelatins, cellular barriers, agarose, alginate, lipid bilayers, polysaccharides) which can comprise a payload (such as, e.g., small molecules, nanotransferosomes, nanoparticles, lipid bodies, liposomes, electrolytes, pharmaceuticals, nutraceuticals and the like) embedded therein (or wherein the nanotransferosomes, lipid bodies, nano-liposomes or liposomes or nano-liposomes themselves are loaded with small molecules, nanoparticles, electrolytes, drugs, pharmaceutical and the like), over which is a micro-porous or a mesoporous substrate comprising an embedded gas or an expandable liquid or expandable gel or gelatin (for example, expandable when heated) for pressure delivery, over which is a PTC heating control, over which is a patch controller.

FIG. 4 graphically illustrates a correlation graph of gelatin viscosity as a function of chemistry for exemplary devices as provided herein.

FIG. 5 schematically illustrates porous channels in exemplary devices as provided herein, which can have an embedded payload or payloads, such as, e.g., small molecules, nanoparticles, nanotransferosomes, lipid bodies, liposomes, electrolytes, pharmaceuticals, nutraceuticals and the like, where the nanotransferosomes, lipid bodies, nano-liposomes or liposomes or nano-liposomes and equivalents can comprise a payload, for example, where the nanotransferosomes, lipid bodies, nano-liposomes or liposomes or nano-liposomes and equivalents are loaded with a small molecule, electrolytes, a biological agent, a pharmaceutical or drug, a nutraceutical or nutraceutical and the like.

FIG. 6 graphically illustrates a characteristic curve to illustrate effective diffusion (as D_{eff} normalized) of payload in exemplary devices as provided herein as a function of pore size in nm.

FIG. 7 graphically illustrates the diffusion tunability based on substrate porosity (nm), temperature (in centigrade) and gelatin viscosity (measured as diffusion rate×1e6); the data demonstrating that the diffusion rate (rate of “payload” delivery out of the device to the skin) is tunable, or is a controllable property of devices as provided herein, and is a function of pressure (not shown in this figure, see FIG. 5), gelatin viscosity, pore size, and temperature; thus, the desired payload delivery amount can be manipulated, or tuned.

FIG. 8, left image graphically illustrates how increases in temperature in the device correspondingly cause an increase in pressure within the device to accelerate or cause the payload in the device (in the layer labeled “gelatin with embedded chemistry” in the illustration of the right image, which schematically illustrates an exemplary product of manufacture as provided herein) to pass out of the device to the skin.

FIG. 9A-B illustrate atomic force microscopy images of 10 μg/mL simvastatin nanotransfersomes imaged at 5 μm (FIG. 9A) and 1 micron (FIG. 9B).

FIG. 10 graphically illustrates the average nanotransfersome vesicle size distribution over sonication intervals at 0, 1, 5, 10, and 20 minutes.

FIG. 11 illustrates electrospray ionization spectra of simvastatin nanotransfersomes loaded in gelatin and microscopy of cold-water fish skin gelatin loaded with simvastatin transfersomes vesicle size; Inset picture shows nanotransfersomes loaded in the gelatin film.

FIG. 12 graphically illustrates data showing the effective permeabilities (Pₑ) of simvastatin (S), simvastatin transfersomes (ST), and simvastatin nanotransfersomes (SNT) for both iso pH skin-PAMPA (Left) and gradient pH skin-PAMPA (Right).

FIG. 13A-B graphically illustrates the ratio of acceptor well concentration [C_{A(t)}] and donor well concentration [C_{D(t)}] of simvastatin, simvastatin transfersomes, and simvastatin nanotransfersomes for both iso pH skin-PAMPA (FIG. 13A) and gradient pH skin-PAMPA (FIG. 13B) over total time of diffusion.

FIG. 14 illustrates the electrospray ionization of standard simvastatin with a parent ion of m/z 419.199 [M+H]⁺ and fragments at m/z 303.118, m/z 285.113, m/z 267.101, m/z 243.106, and m/z 225.093.

FIG. 15A-E illustrate Liquid chromatography-mass spectrometry of standard simvastatin for quantitation of in vivo simvastatin blood plasma concentrations (FIG. 15A) simvastatin standard at 10 ng/mL concentration, (FIG. 15B) simvastatin standard at 100 ng/mL concentration, (FIG. 15C) simvastatin standard at 1 μg/mL concentration, (FIG. 15D) simvastatin standard at 10 μg/mL concentration, (FIG. 15E); Calibration curve generated from simvastatin standards with a 0.9997 goodness of fit.

FIG. 16 graphically illustrates average simvastatin blood plasma concentrations for both female and male rats at time intervals of 0, 6, 24, and 48 hours after transdermal patch administration.

Like reference symbols in the various drawings indicate like elements.

### DETAILED DESCRIPTION

In alternative embodiments, provided are products of manufacture and methods for using them, for the transdermal or transmucosal delivery of any compound or composition such as a pharmaceutical, an electrolytes, a natural product or a nutraceutical. In alternative embodiments, provided are products of manufacture that utilize a nanoporous substrate coupled with controlled melt or solubilization of polymers such as gels or gelatins (e.g., a fish skin gelatin film, or equivalent) for the delivery of the compounds or compositions, active agents and electrolytes transdermally or transmucosally.

In alternative embodiments, products of manufacture as provided herein comprise a so-called Nano-Encapsulated Transdermal (NET) system, which comprises use of a microporous polyolefin silica substrate, or equivalent, soaked with or being layered with a fish skin gelatin film, or equivalent, for the transdermal or transmucosal delivery of any composition or compound such as a pharmaceutical or a nutraceutical, or an electrolyte.

In alternative embodiments, products of manufacture as provided herein (e.g., the NET system) are sewn into or onto or attached to bands, eyeglasses, watches, clothes, shoes, hats or any garment or wearable, or bandages or patches, or worn or applied as a patch or bandage onto the skin or a mucous membrane. In alternative embodiments, the products of manufacture as provided herein (e.g., the NET system) as provided herein comprises an adhesive such that the product of manufacture can be attached or reversibly adhered to a skin or a mucous membrane.

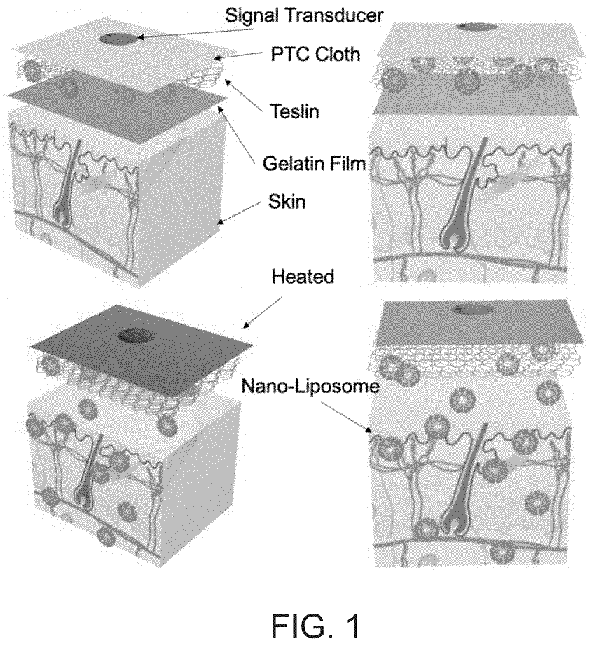

An exemplary product of manufacture as provided herein, a so-called NET system, is illustrated in FIG. 1, which shows an exemplary patch applied to the surface of the skin, where applied heat causes the payload-containing nanoliposomes to permeate through the gelatin layer and onto or into the skin.

In alternative embodiments, exemplary products of manufacture comprises at least three layers:

a first, an inner layer, or layer meant to be approximate to the skin, comprising a heat sensitive porous material, wherein the heat sensitive porous material becomes porous, semi-solid or soluble at a temperature of between about 50° C. to 68° C., between about 55° C. to 65° C., or at about 60° C., and optionally the heat sensitive porous material comprises a gelatin film (e.g., a fish gelatin), or equivalent; and optionally the heat-sensitive porous material (e.g., the fish skin gelatin film) or equivalent is layered on a microporous polyolefin silica substrate (e.g., by PPG Industries, Pittsburgh, Pa.), or equivalent, and optionally the heat-sensitive porous material or equivalent is layered on a microporous polyolefin silica substrate or equivalent;

a second, an inner (or middle) layer comprising a plurality of nanotransferosomes, lipid bodies, nano-liposomes, liposomes or nano-liposomes or equivalent particles comprising or having contained therein a payload, wherein the payload can be any compound, composition, payload or active agent, including for example, a small molecule, a protein or peptide, a polysaccharide, a lipid, an ion, a reagent, a pharmaceutical, an electrolyte, a natural product and/or a nutraceutical, and the payload-comprising nanotransferosomes, lipid bodies, nano-liposomes, liposomes or nano-liposomes or equivalent particles are embedded in a microporous polyolefin silica substrate (e.g., by PPG Industries, Pittsburgh, Pa.), or equivalent; and

a third, an outer layer comprising or substantially comprising a Positive Temperature Coefficient (PTC) material (e.g., by PPG Industries), e.g., a poly-crystalline ceramic material such as a microporous polyolefin silica substrate, which can be made semi-conductive by a dopant, wherein optionally the PTC material is printed onto, attached to or embedded on a polymer (e.g., polyester or polyethylene terephthalate (PET), or acrylic-comprising polymer), or a textile, a fabric or a cloth.

In alternative embodiments, the second layer of the at least three layers of an exemplary product of manufacture comprises a plurality of nanotransferosomes, lipid bodies, nano-liposomes, liposomes or nano-liposomes or equivalent particles, e.g., at a concentration of about 6 mg/cc. For example, the amount or concentration of nanotransferosomes, lipid bodies, nano-liposomes, liposomes or nano-liposomes or equivalent particles in the middle or inner layer can be a maximum of about two, three or four 2 mg doses, which for example can be provided to the first responder or patient suffering from the chemical warfare agent (CWA), or any toxic agent or toxic gas exposure.

In alternative embodiments, the nanotransferosomes, lipid bodies, nano-liposomes, liposomes or nano-liposomes or equivalent particles are only designed or intended to be released once through the inner porous, or fish skin gelatin, layer, or equivalent, or the nanotransferosomes, lipid bodies, nano-liposomes, liposomes or nano-liposomes or equivalent particles can be repeatedly released in a time regulated, pulsed manner by repeated active heating and active or passive cooling of the inner heat sensitive porous material (e.g., gelatin) layer.

In alternative embodiments, the product of manufacture, particularly, the inner heat-sensitive porous layer, is heated to at least about 60° C., or to between about 55° C. to 65° C., thus (substantially or partially) melting or solubilizing the heat-sensitive porous layer and allowing permeation of the 1 nanotransferosomes, lipid bodies, nano-liposomes, liposomes or nano-liposomes or equivalent particles with their payload through the heat-sensitive porous layer (e.g., the gelatin) and onto or into the skin or mucous membrane.

In alternative embodiments, when applying and using the products of manufactures as provided herein (e.g., the so-called NET systems), the heat applied to the PTC material, and thus the heat-sensitive porous layer, is limited to avoid irritation or inflammation, or scalding or burning, of the skin.

In alternative embodiments, the payload comprises any composition or active agent, including for example a small molecule, protein or peptide, a nucleic acid, a lipid, a polysaccharide, a pharmaceutical, a natural product or a nutraceutical, an electrolyte or ion, or any reagent, for example, the payload can comprise a CWA, or toxic gas or agent, antidote, e.g., as a nano-encapsulated atropine or agent as described in US 2019 0119237 A1.

In alternative embodiments, the nanotransferosomes, lipid bodies, nano-liposomes, liposomes or nano-liposomes or equivalent particles can be made of any known materials, and the payload can be loaded onto or into the nanotransferosomes, lipid bodies, nano-liposomes, liposomes or nano-liposomes or equivalent particles using any known technique, e.g., as described by Demirci et al, Nanoencapsulation Technologies for the Food and Nutraceutical Industries 2017, Pages 74-113; or Nomani et al Int J Adv Pharmacy Med Bioallied Sci. Vol. 2016 (2016), Article ID 92, 1-10; or as described in U.S. pat app nos. 20110165068 or 20090263473; or U.S. Pat. Nos. 10,272,041; 10,179,106 or 8,685,440. In alternative embodiments, the nanotransferosomes, lipid bodies, nano-liposomes, liposomes or nano-liposomes or equivalent particles comprise at least one lipid bilayer comprising for example: phosphatidylcholine (PC) and dipalmitoyl PC, lecithin or esterified lecithin, dipalmitoyl-phosphatidyl-choline (DPPC), 1,2-distearoyl-sn-glycero-3-phospho-ethanolamine (DSPE), sphingomyelin, N-[1-(2,3-Dioleoyloxy)propyl]-N,N,N-trimethylammonium methyl-sulfate (DOTAP), 1,2-dimyristoyl-sn-glycero-3-phosphocholine (DMPC), 1,2-dimyristoyl-sn-glycero-3-phospho-(1′-rac-glycerol) (DMPG), soy hydrogenated L-α-phosphatidylcholine (HSPC), cholesterol, 1,2-distearoyl-sn-glycero-3-phospho-(1′-rac-glycerol) (DSPG), 1,2-dioleoyl-sn-glycero-3-phosphocholine (DOPC), 1,2-dipalmitoyl-sn-glycero-3-phospho-(1′-rac-glycerol) (DPPG), 1,2-distearoyl-sn-glycero-3-phosphocholine (DSPC), phospholipids (e.g., from hen eggs), soybean oil or polysorbate 80 or polyoxyethylene (20) sorbitan monooleate.

In alternative embodiments, products of manufactures as provided herein (e.g., the so-called NET systems) comprise use of a fish skin gelatin or equivalent in the first, or heat-sensitive porous layer, because this gelatin (or heat sensitive porous material) becomes soluble at about 60° C., thus allowing the payload-comprising nanotransferosomes, lipid bodies, nano-liposomes, liposomes or nano-liposomes or equivalent particles to diffuse through the first, or porous layer and interact with the skin, while at temperatures below about 50° C. the fish skin gelatin or equivalent solidifies, preventing the nanotransferosomes, lipid bodies, nano-liposomes, liposomes or nano-liposomes or equivalent particles from diffusing out of the product of manufacture. This heat modulating opening and closing of pores, or solubilization and solidifying of the heat sensitive porous material or skin gelatin layer, and can allow for multiple doses to be administered without the need of a second or third application, or for need for a syringe injection; this can reduce the chances of infection in the first responders.

In alternative embodiments, products of manufacture as provided herein (e.g., the so-called NET systems) comprise a heating module or device, for example, a heating device or module can be attached to or can be implanted in the products of manufacture.

In alternative embodiments, the products of manufacture as provided herein (e.g., the so-called NET systems) further comprise an ultra-thin magnetic spiral antenna and a near-field communication (NFC) chip, which are attached to or are embedded into or onto a section of the product of manufacture, wherein the ultra-thin magnetic spiral antenna is operatively connected to the near-field communication (NFC) chip, wherein the ultra-thin magnetic spiral antenna can receive a remote electromagnetic signal and transmit the signal to the near-field communication (NFC) chip, and the NFC chip is operatively connected to the heating module to further transmit the signal and result in heating of the heat sensitive porous material of the inner later of the product of manufacture to a temperature of between about between about 50° C. to 68° C., between about 55° C. to 65° C., or at about 60° C.

In alternative embodiments, products of manufactures as provided herein (e.g., the so-called NET systems) comprise use of a Near Field Communication (NFC) chip attached implanted in or attached to the products of manufacture; the NFC chip can be controlled by a user using a signal transducer controlled by a remote device, for example, by using a mobile phone or smartphone, computer or any remote signaling device. In alternative embodiments, the NFC chip is operably linked or connected to the heating device such that the NFC chip is used to turn the heating device or module on or off, wherein the heating device or module is implanted in or attached to the products of manufacture.

Alternatively, the heating device can be controlled by a manual control such a switch or touch sensitive pad mounted on the product of manufacture.

In alternative embodiments, to permeate the skin passively the nanotransferosomes, lipid bodies, nano-liposomes, liposomes or nano-liposomes or equivalent particles must be below about 30 nm in diameter. Thus, in alternative embodiments, products of manufactures as provided herein (e.g., the so-called NET systems) utilize nanotransferosomes, lipid bodies, nano-liposomes, liposomes or nano-liposomes or equivalent particles averaging less than about 25 nm in diameter, or no more than about 25 nm in diameter, to ensure optimal transdermal diffusion.

In alternative embodiments, the outer layer comprising a microporous polyolefin silica substrate (e.g., as made by PPG Industries) have pores no larger than about 30 nm diameter to ensure that no large nanotransferosomes, lipid bodies, nano-liposomes, liposomes or nano-liposomes or equivalent particles can be trapped in the substrate, allowing for optimal efficiency. At this pore size a pore volume of 30% can be achieved using SP1000, allowing for flexibility of a cloth or fabric to be retained while maintaining sufficient storage volume for nanotransferosomes, lipid bodies, nano-liposomes, liposomes or nano-liposomes or equivalent particles, which illustrates different pore volumes based on pore size of each microporous polyolefin silica.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications.

Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

### Example 1: Comparison of In Vitro Transdermal Parallel Artificial Membrane Permeability Assay (PAMPA) with In Vivo Techniques for the Delivery of Simvastatin-Encapsulated Nanotransfersomes

This example demonstrates making and using exemplary products of manufacture as provided herein, in particular, this example describes making and using an exemplary gelatin patch loaded with nanotransfersomes having a small molecule payload, which in this example is the small molecule simvastatin.

Simvastatin, a common cardiovascular medication prescribed to lower Low-Density Lipoprotein (LDL) cholesterol levels in humans, has low bioavailability when administered orally. To increase the bioavailability of simvastatin using a passive transport mechanism, simvastatin nanotransfersomes were generated to promote transdermal drug delivery. These nanotransfersomes were subjected to in vitro analytical techniques using Parallel Artificial Membrane Permeability Assays (PAMPA) and in vivo techniques using Long Evans rats. The samples were then analyzed using Electrospray Ionization-Mass Spectrometry (ESI-MS). When determining the transdermal capability of simvastatin nanotransfersomes using in vitro analytical techniques, the percentage of diffused simvastatin was higher than the in vivo analysis being 20.1% after 6 hours in vitro, while only 0.01318% for females and 0.0079% for males at the 48-hour interval in vivo. While in vitro analyses are more cost-effective and less labor-intensive, studies should continue to utilize both in vitro and in vivo analysis for confirmation of successful transdermal drug delivery.

Nanotransfersomes encapsulating simvastatin were developed, their transdermal capabilities were monitored using the skin-PAMPA technique. Simvastatin nanotransfersomes were fabricated in a fish skin gelatin on a synthetic paper TESLIN® substrate. We characterized their in vivo permeability on Long Evans female and male rats. Their permeabilities were then analyzed to determine the efficacy of delivery of the nanotransferosomes in the in vitro (PAMPA) assay compared to the in vivo study.

### Methods

### Liposome Formation

The liposomes were formed using the thin-film lipid cake method to form a concentration of 20 mg/mL of simvastatin (Fisher Scientific, Hampton, N.H., USA) encapsulated liposomes. Specifically, 10 mL of 95% v/v ethanol (Sigma Aldrich, St. Louis, Mo., USA) was added into a Round Bottom Flask (RBF) in a water bath heated to 40° C. with stirring. Phosphatidylcholine (PC) 92% (Spectrum Chemical Mfg Corp, New Brunswick, N.J., USA), 750 mg, was first added and dissolved in the ethanol. Once dissolved, 300 mg of cholesterol (Fisher Scientific, Hampton, N.H., USA) and 300 mg of Kolliphor RH40™ (Sigma Aldrich, St. Louis, Mo., USA) were added to stabilize the liposomes allowing them to become transfersomes. Once dissolved, 200 mg of simvastatin was added to the RBF and allowed to dissolve in the ethanol. The ethanol was then evaporated under reduced pressure at 40° C. The remaining thin lipid film was then allowed to come to room temperature. Once at room temperature, 10 mL of 5 mM ammonium bicarbonate (Fisher Scientific, Hampton, N.H., USA) buffer in 18.2 microohm (mΩ) H₂O was added, and the RBF was shaken vigorously for 20 minutes to bring a final concentration of 20 mg/mL simvastatin encapsulated transfersomes. The solution was imaged using a TE-2000™ inverted microscope (Nikon, Melville, N.J., USA) to determine the size of the multi-lamellar transfersomes.

### Sonication Parameters

The transfersomes were then subjected to sonication, reducing their size from micrometers to nanometers using a 705 SONIC DISMEMBRATOR™ (Fisher Scientific, Hampton, N.H., USA). The transfersomes were subjected to 50% intensity over 20 minutes with pulse sonication of 10 seconds on and 15 seconds off. A 1 μL aliquot of sample was extracted from the 10 mL at the 0, 1, 5, and 10-minute intervals of sonication and sized using the same microscopy as above.

### Atomic Force Microscopy

A CoreAFM™ (NanoSurf, Liestal, Switzerland) was utilized to determine the size of nanoparticles sub 300 nm. Samples were prepared at a 1 mg/mL concentration of simvastatin nanotransfersomes in 18.2 mΩ water and were further diluted to 10 μg/mL in 18.2 mΩ water prior to analysis. Freshly cleaved mica (Muscovite Mica, Vi, Nialco, Japan) was used for the substrate to place the sample on and AFM images were collected using Dynamic force mode. The scanning tips were Si, N-type, gold-coated with a length of 225 μM×40 (Appnano, Mountain View, Calif., USA). The system was set up using 512 scans per line at a scan speed of 1.5 s per line over a 5 μm section. Once completed, a smaller 1 μm section was focused on and analyzed using the same parameters. The particle size of the vesicles was calculated according to previous reports⁴¹.

### Encapsulation Efficiency

Encapsulation efficiency was monitored by removing 1 mL of sonicated nano-transfersomes. The 1 mL was then centrifuged at 3000 g for 20 minutes using a Sorvall Legend Micro 17™ centrifuge (ThermoFisher Scientific, Waltham, Mass., USA) to obtain a pellet of the nano-transfersomes. The supernatant was then removed, and the pellet was reconstituted in optima grade chloroform:methanol 50:50 with 0.1% ammonium acetate (Fisher Scientific, Hampton, N.H., USA) as a charge carrier. The sample was then diluted to 10 μg/mL and analyzed using electrospray ionization mass spectrometry using a Waters Synapt G2-Si™ mass spectrometer (Waters Corporation, Milford, Mass., USA).

### Certramide Synthesis

To mimic the transdermal barrier using Parallel Artificial Membrane Permeability Assay (PAMPA), a C12/C18 certramide was synthesized following the previously reported method⁴¹. Briefly, 97% diacetyl-L-tartaric anhydride (Fisher Scientific, Hampton, N.H., USA) (25 mmol) was placed in an RBF with 25 mmol octadecylamine 97% (Fisher Scientific, Hampton, N.H., USA) and 40 mL of HPLC tetrahydrofuran (THF) (Fisher Scientific, Hampton, N.H., USA) and left to stir overnight at room temperature. The THF was then removed under reduced vacuum and 45 mL of 99% thionyl chloride (Fisher Scientific, Hampton, N.H., USA) was added to the RBF along with 0.7 mL of ACS pyridine (Fisher Scientific, Hampton, N.H., USA). The RBF was then placed in a 70° C. water bath and left to react for 15 min. The thionyl chloride was then evaporated off and purified over activated charcoal in 15 mL of 99.6% dichloromethane (DCM) (Fisher Scientific, Hampton, N.H., USA). The DCM was then removed under reduced pressure. The remaining semi-solid product was dissolved in 90 mL of ethanol and 20 mL of 99% acetyl chloride (Fisher Scientific, Hampton, N.H., USA). The mixture was stirred for 24 hours and the white precipitate was filtered and washed. The precipitate was then treated with 8 mmol of dodecylamine 98% (Fisher Scientific, Hampton, N.H., USA) in 50 mL of ACS grade xylene (Fisher Scientific, Hampton, N.H., USA) at 130° C. in an oil bath. The reaction was stirred for 24 hours and then filtered and washed. Once completed, a 60:20:20 ratio of certramide (C12-C18), 95% cholesterol (Fisher Scientific, Hampton, N.H., USA), and 97% stearic acid (Fisher Scientific, Hampton, N.H., USA) were mixed in chloroform at a 20 mg/mL concentration.

### Skin-PAMPA

A Millipore™ multiscreen 96-well assay (MAIPNTR10) and a Multiscreen transport receiver plate (MATRNPS50; Fisher Scientific, Waltham, Mass.) was used. The Parallel Artificial Membrane Permeability Assay (PAMPA) membrane was coated with 10 μL of the lipid mixture from above and allowed to evaporate. A 200 μL aliquot of 1 mg/mL in 10 mM ammonium bicarbonate, pH 6.4, simvastatin nano-transferomes was placed in the PAMPA well donor well. A 200 μL aliquot of 10 mM ammonium bicarbonate, pH 7.4, was then added to the acceptor well. In a separate PAMPA well, a 200 μL aliquot of 1 mg/mL in 10 mM ammonium bicarbonate, pH 7.4, simvastatin nano-transferomes was placed in the PAMPA well donor well with 200 μL of 10 mM ammonium bicarbonate, pH 7.4, was added to the acceptor well. The wells were then incubated for 6 hours to determine the permeability of simvastatin nano-transfersomes in vitro. Samples were made for 1, 2, 3, 4, 5, and 6 hours to monitor the concentration of simvastatin nano-transfersomes over time.

### PAMPA Equations

Calculations for the determination of transdermal permeability were performed according to the permeability across a transdermal membrane when subjected to a membrane retention under gradient-pH conditions⁴³.

$${P}_{e} = - ( \frac{2.303 \cdot {V}_{D}}{A \cdot ( t - {τ}_{LAG} )} ) \cdot ( \frac{1}{1 + {r}_{a}} ) {log}_{10} [ - {r}_{a} + ( \frac{1 + {r}_{a}}{1 - R} ) \cdot ( \frac{{C}_{D} ( t )}{{C}_{D} ( 0 )} ) ] \qquad ( 1 )$$

Where Pₑ is the effective permeability coefficient (cm/s), A is the filter area (0.3 cm²), V_{D} and V_{A} are the volumes of the donor and acceptor well, respectively, t is the incubation time, τ_{LAG} is the time to reach steady-state, C_{D}(t) is the concentration of the compound in the donor phase at time t (mol/cm³), C_{D}(0) is the concentration of the compound in the donor phase at time 0 (mol/cm³), C_{A}(t) is the concentration of the compound in the acceptor well at time t (mol/cm³), rₐ is the sink asymmetry ratio (gradient-pH-induced):

$${r}_{a} = ( \frac{{V}_{D}}{{V}_{A}} ) \cdot \frac{{P}_{{e}_{( A \to D )}}}{{P}_{{a}_{( D \to A )}}} \qquad ( 2 )$$

The membrane retention factor is R and rₐ is the sink asymmetry ratio (gradient-pH-induced).

$$R = 1 - ( \frac{{C}_{D} ( t )}{{C}_{D} ( 0 )} ) - ( \frac{{V}_{A}}{{V}_{D}} ) \cdot ( \frac{{C}_{A} ( t )}{{C}_{D} ( 0 )} ) \qquad ( 3 )$$

### In Vivo Study

To determine the in vivo permeability of the simvastatin nano-transfersomes, 9 adult Long Evans rats (Rattus novegicus), 6 males and 3 females, ranging from 9-12 months, were dosed with an exemplary product of manufacture as provided herein, in particular, an exemplary gelatin patch loaded with nanotransfersomes. The Long Evans rats were stored and maintained at the University of North Texas Vivarium in standard shoebox caging. The temperature was maintained at 18° C.-25° C. with natural ventilation. Rats had access to commercially available rat pellets and water ad libitum throughout the study period. All animal protocols were approved by the University of North Texas Institutional Animal Care and Use Committee and conformed to the Guide for the Care of Use of Laboratory Animals published by the US National Institute of Health (NIH Publication No. 82-23, revised 1996).

To prepare the exemplary transdermal patch, gelatin from cold-water fish skin (Sigma Aldrich, St. Louis, Mo., USA) was weighed at 4% (w/v) ratio dissolved in 18.2 mΩ H₂O with stirring and heated at 45° C. for 30 min. Once dissolved, 99% glycerol (Fisher Scientific, Hampton, N.H., USA) at 25% (w/w) based on fish skin gelatin weight was added. Once completed, an equal volume of nanotransfersomes was added to the fish skin gelatin solution giving a final concentration of 10 mg/mL of simvastatin nanotransfersomes. Once the gelatin solution was complete, 1.2 mL of the gelatin solution was placed on the paper substrate, TESLIN®, and evaporated overnight. The patches were then placed in-between the shoulder blades of shaved rats, 3 males and 2 females, keeping the remaining 3 males and 1 female rat as control samples. The patches were placed on the rats for 48 hours and 0.2 mL of blood was collected using the tail-vein method at 0, 6, 24, and 48 hours. After blood collection, the blood was centrifuged at 1500 rpm for 15 min at RT. The simvastatin was extracted from the blood plasma by placing 100 μL of plasma in 1.25 mL of tert-butyl methyl ether 99.9% (Fisher Scientific, Hampton, N.H., USA) and was vigorously shaken for 15 min. The solution was then centrifuged at 4000 rpm for 10 min at RT. The supernatant was extracted and evaporated. Once evaporated, the sample was reconstituted in 200 μL of mobile phase.

### LC-MS

A Waters ACQUITY UPLC™ (Waters Corporation, Milford, Mass., USA) and Waters SYNAPT G2-Si™ mass spectrometer (Waters Corporation, Milford, Mass., USA) were used to determine the concentration simvastatin in the blood samples obtained from the Long Evans rats. The experiment used a binary solvent method consisting of 18.2 mΩ water containing 0.1% formic acid (solvent A) and acetonitrile with 0.1% formic acid (solvent B). The initial flow of the binary pump was set to 60% solvent A and 40% solvent B at a flow rate of 0.380 mL/min. A gradient changed to 10% solvent A with 90% solvent B at a linear gradient over a period of 3 minutes and held for 3.30 minutes. The column was then reconditioned back to the original condition over a period of 3.4 minutes at a linear gradient. A 20 μL sample of extracted rat blood plasma was injected to an Agilent POROSHELL 120™, C18, 2.7 μm, 4.8×50 mm column (Agilent Technologies, Santa Clara, Calif., USA) set at 35° C. The mass spectrometer analyzed masses within the m/z 50 to 500 range. The source temperature and desolvation gas set point was set to 80° C. and 500 L/h, respectively, with a capillary voltage set at 3 kV.

### Results

### Nanotransfersomes Characterization

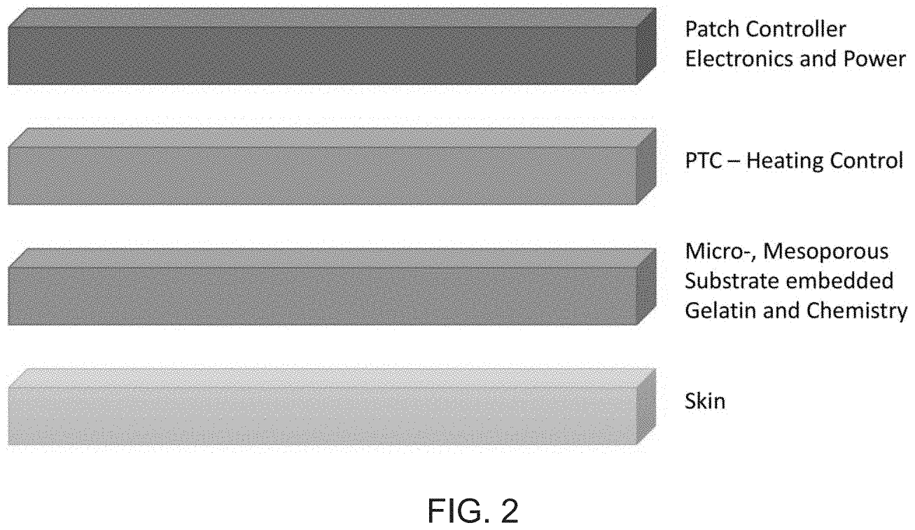

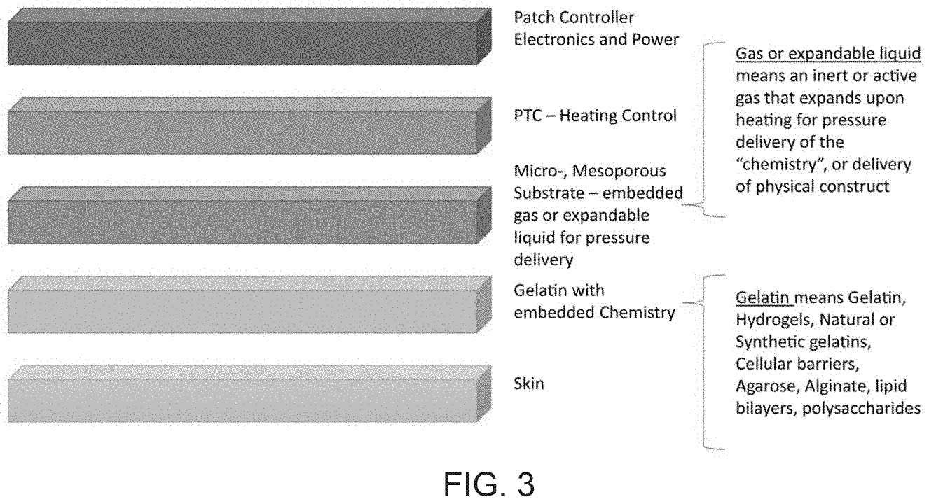

After the addition of water and sonication, the multilamellar transfersomes were imaged and plotted using a Moore's plot for characterization. The size of the transfersomes prior to sonication was 825.2+/−50.2 μm. After 1 minute of sonication time, the size of the transfersomes was 150.4+/−37.8 μm. After 5 minutes, the size of the transfersomes was found to be 41.8+/−6.1 μm, and after 10 minutes, the size of the nanotransfersomes was 338 nm+/−42.7 nm. Moreover, after 20 minutes of sonication time, the nanotransfersomes were found to be 12.69+/−5.53 nm in diameter (FIG. 1). The data was plotted, and a curve fitting found that after 15.36 minutes the average size of the nanotransfersomes is <100 nm (FIG. 2), allowing for greater passive diffusion across the transdermal barrier. The encapsulation efficiency of the simvastatin nano-transfersomes was found to be 89.68% encapsulation, leading to a final concentration of 17.936 mg/mL encapsulated simvastatin (FIG. 3).

### Skin-PAMPA

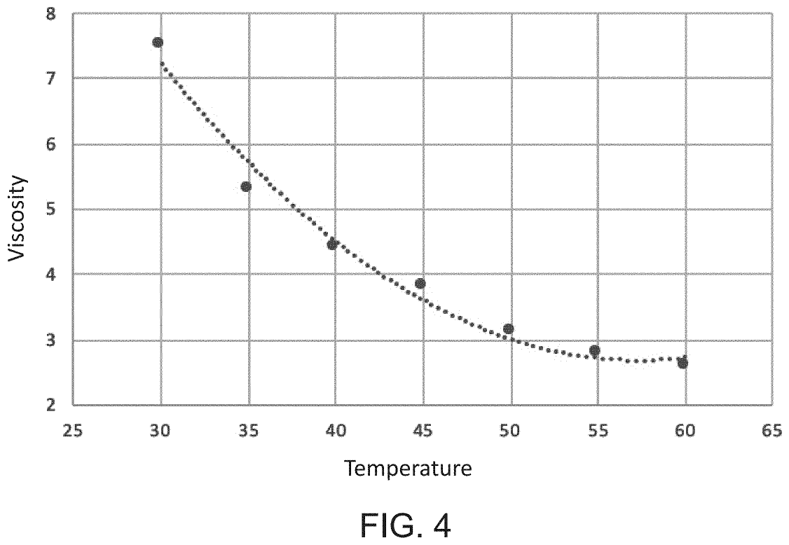

To confirm the Skin-PAMPA analysis was functional, simvastatin, simvastatin transfersomes, and simvastatin nanotransfersomes were utilized to determine the permeability across a transdermal membrane while incorporating membrane retention under a gradient-pH factor to generate sink conditions. The apparent permeability of the simvastatin crossing the iso-gradient donor and acceptor well was 6.001×10⁻⁷ (cm/sec) and the apparent permeability of the simvastatin transfersomes was 1.793×10⁻⁶ (cm/sec) (FIG. 4). The transfersomes will allow the simvastatin to interact with the skin membrane, but still allow a slightly better diffusion rate. However, once the simvastatin nanotransfersomes are generated through sonification, the apparent permeability increased to 1.596×10⁻⁵ (cm/sec). The skin-PAMPA is slightly more acidic than the rest of the body, generating ionic particles that will prevent further diffusion across the non-ionic lipid membrane. The apparent permeability for all of the drug conditions, simvastatin free drug, simvastatin transfersomes, and simvastatin nanotransfersomes decreased to 7.985×10⁻⁸, 5.005×10⁻⁷, and 8.285×10⁻⁶ (cm/sec), respectively, following the expected trend.

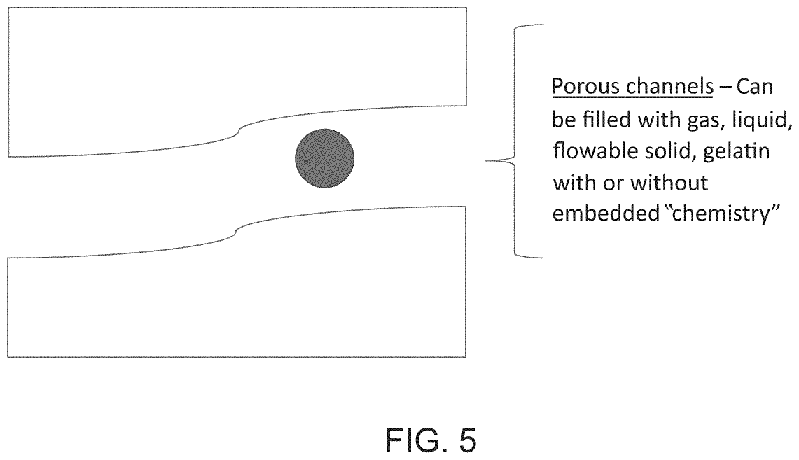

In order to determine how the kinetics of simvastatin may have changed over time, a percentage of the concentration of the simvastatin in the acceptor well over the concentration of the donor well was performed at each hour interval mark (FIG. 5). The iso-pH conditions resulted in simvastatin free drug permeating across the skin-PAMPA at 0.08% diffusion of simvastatin at the 1-hour interval mark and increased to 1.2% diffusion of simvastatin at the 6-hour interval (FIG. 5A). The simvastatin transfersomes had slightly better permeability over time with the iso-pH conditions starting at 0.6% at the 1-hour interval, increasing to 3.5% diffusion across the skin-PAMPA barrier (FIG. 5A). The iso-pH skin-PAMPA conditions for the simvastatin nanotransfersomes resulted in 2.3% diffusion at the 1-hour interval and increased up to 29.7% at the 6-hour interval (5A). The diffusion of simvastatin free drug across the pH gradient sink conditions started at 0.04% diffusion of simvastatin at the 1-hour interval mark and increased to 0.2% diffusion at the 6-hour interval (FIG. 5B). The pH gradient of the simvastatin transfersomes started at 0.3% at the one-hour interval and increased to 0.9% at the 6-hour interval. The simvastatin nanotransfersomes had the best permeability across the skin-PAMPA membrane among all three experimental groups (FIG. 5B). Additionally, the pH gradient sink conditions resulted in 1.6% diffusion across the membrane at the 1-hour interval and 20.1% diffusion at the 6-hour interval (FIG. 5 B).

### LC-MS Validation

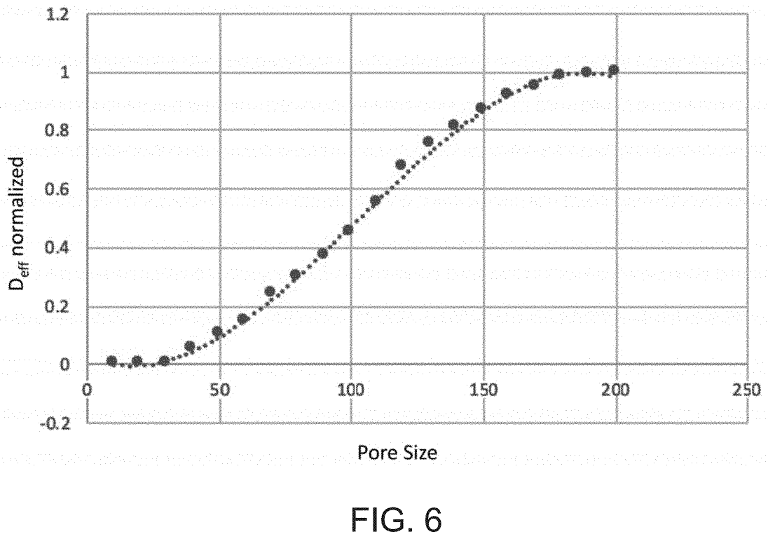

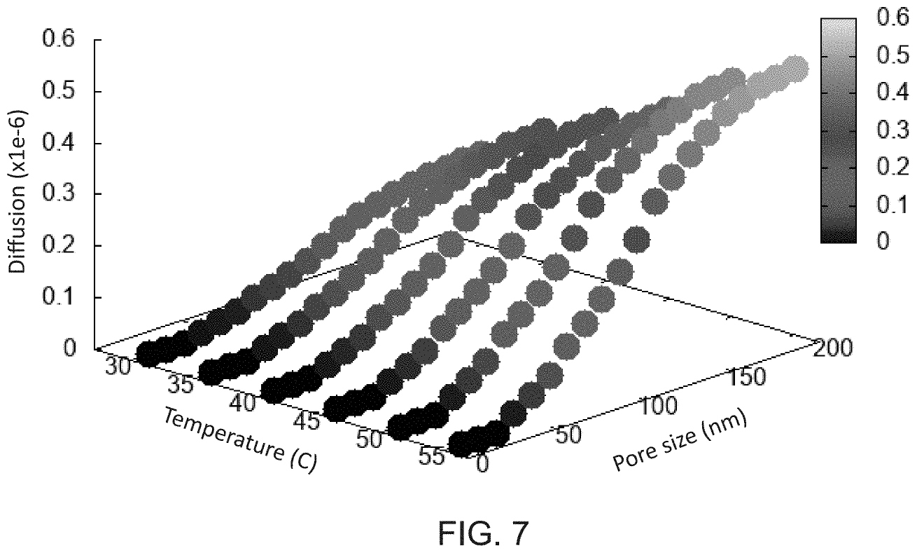

Standards of simvastatin were prepared at 10 ng/mL, 100 ng/mL, and 1 μg/mL to provide a working range for the in vivo study. The calibration curve was performed to confirm that the simvastatin eluted off the column after 5.019 minutes with the proper mass spectral peaks at m/z 419.199 [M+H]⁺, m/z 303.118, m/z 285.113, m/z 267.101, m/z 243.106, and m/z 225.093 (FIG. 6). The calibration curve was analyzed prior to analyzing the blood samples and provided a goodness of fit value of 0.99973274 (FIG. 7).

### In Vivo Analysis

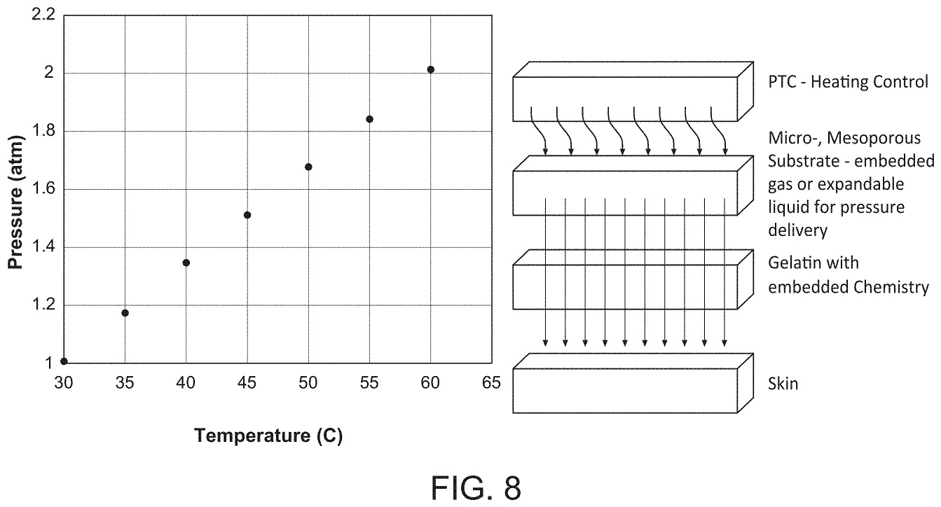

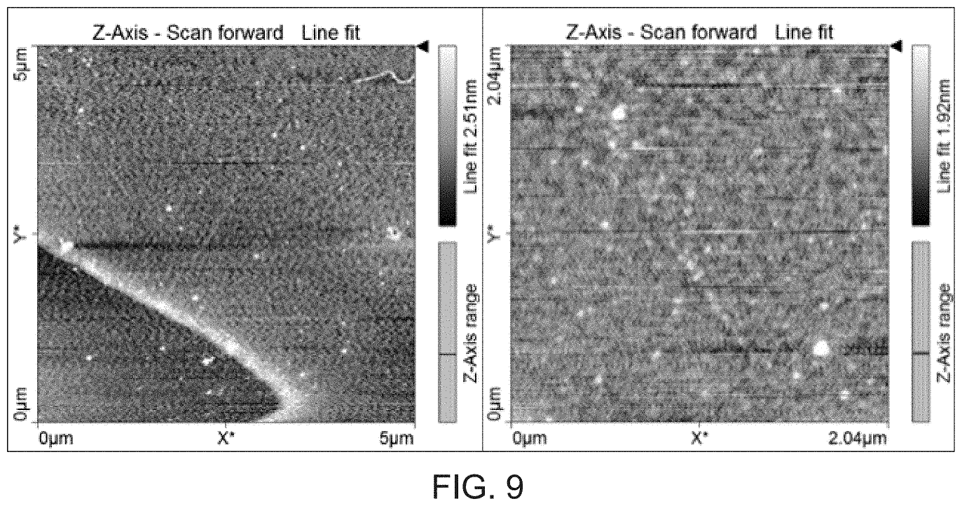

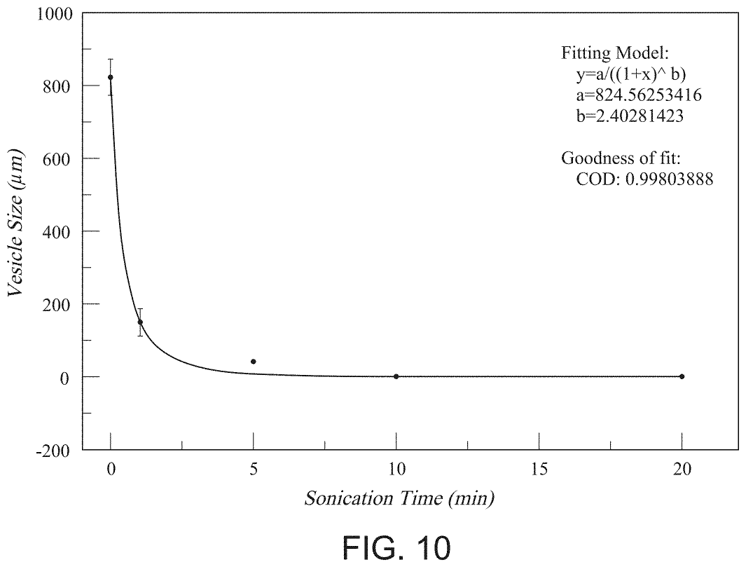

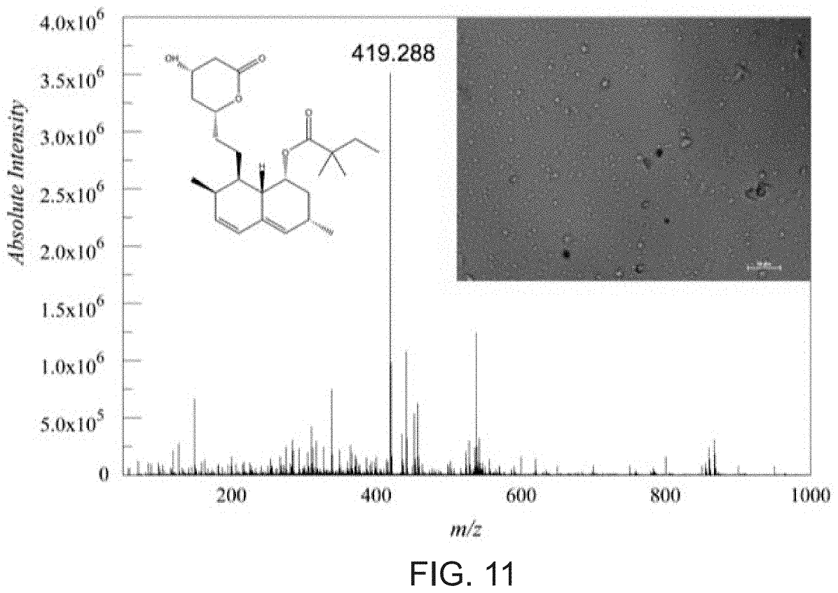

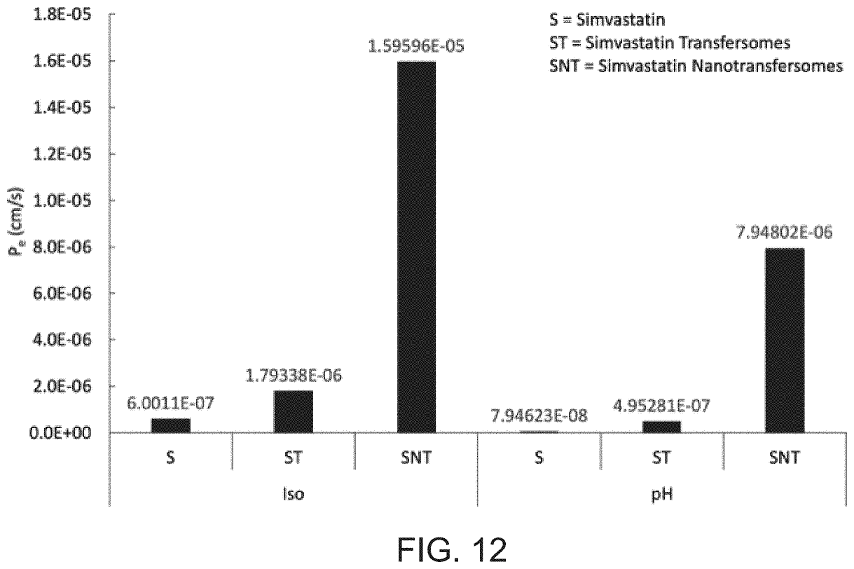

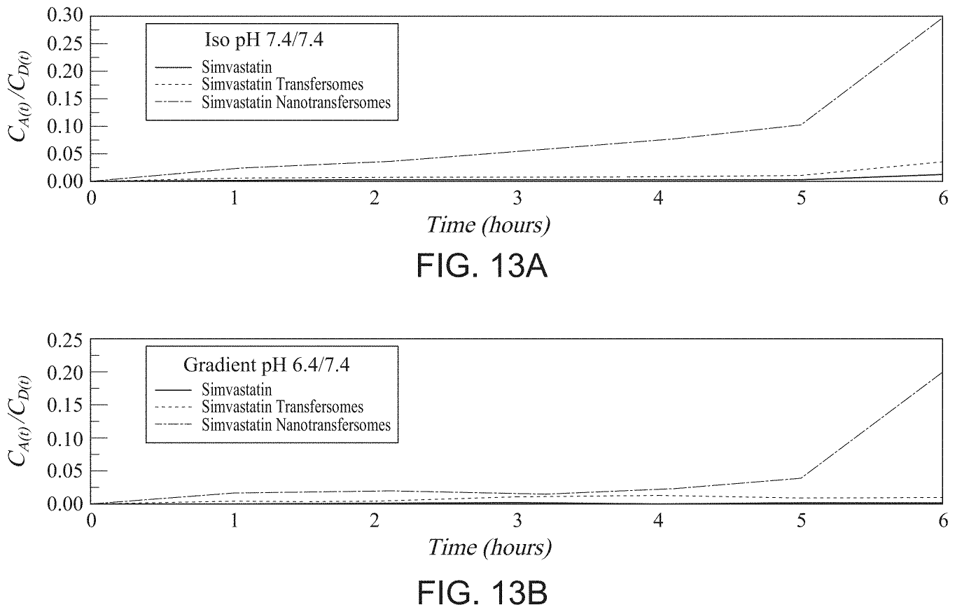

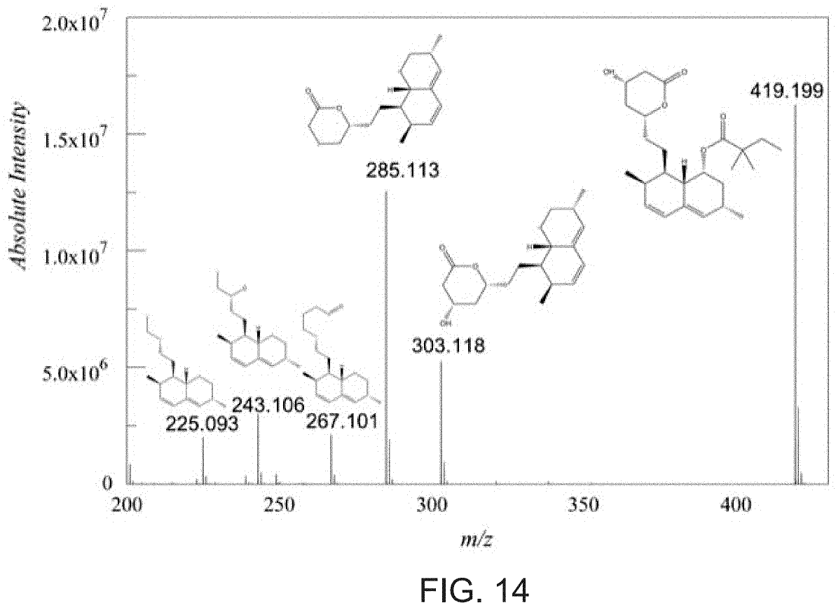

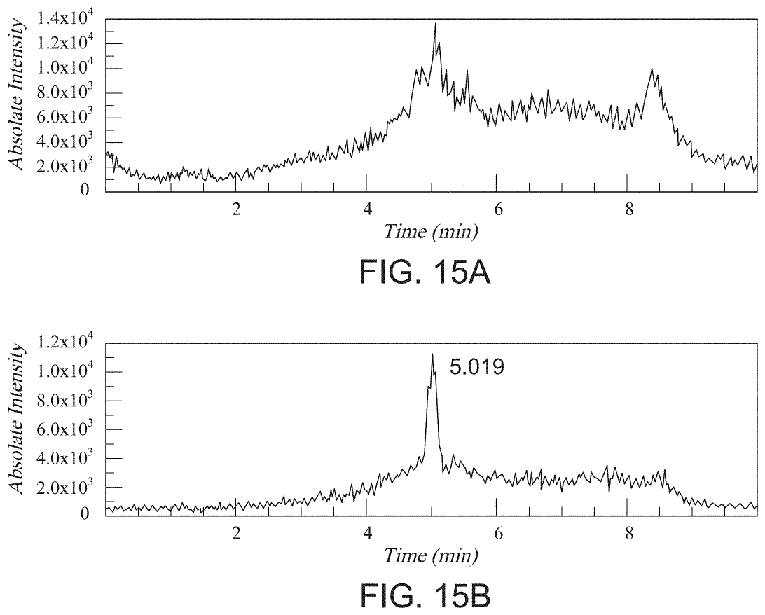

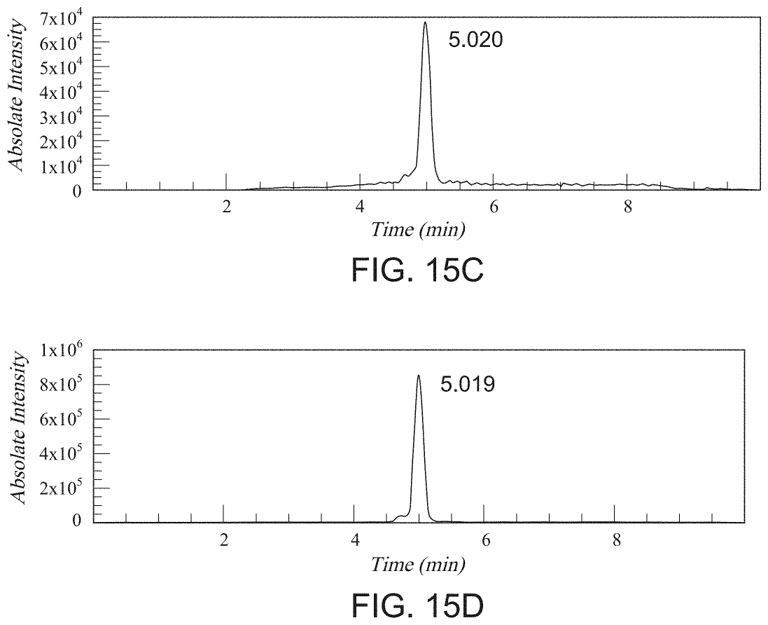

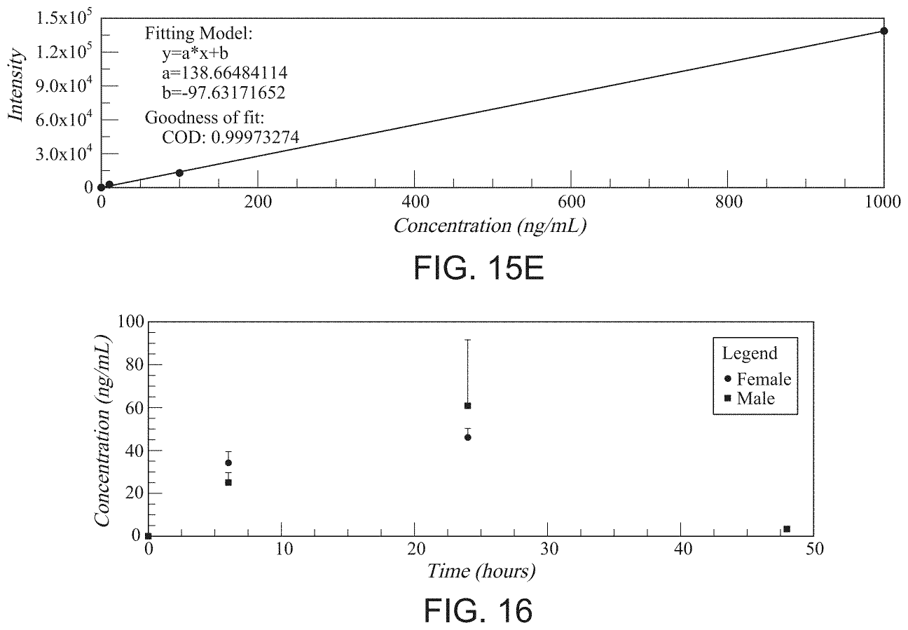

A total of 3 female rats (F1, F2, F3) and 6 male rats (M1-M6), were used for the in vivo study. F1, M2, M4, and M6 were used as control rats and did not receive any transdermal patch treatment. F2, F3, M1, M3, and M5 all received the simvastatin nanotransfersomes. At the time 0 interval, as expected, there was no simvastatin present in the blood from any of the control or simvastatin-treated rats. On average, the females had a higher concentration of simvastatin in the blood plasma than males at 6 hours following patch placement; being 34.25+/−5.23 ng/mL compared to 25.16+/−4.69 ng/mL in males (FIG. 8). However, the males had a higher concentration at the 24-hour period with an average concentration of 60.85+/−31.16 ng/mL compared to 46.103+/−4.26 ng/mL in the female rats (FIG. 8). The large standard deviation in the male rats at the 24-hour interval is found in M3, having a simvastatin blood plasma concentration of only 26.775 ng/mL, while the other two experimental rats had simvastatin blood plasma concentrations of 87.886 ng/mL and 67.905 ng/mL. At the 48-hour interval, both males and females had similar simvastatin blood plasma concentrations being 3.18+/−0.515 ng/mL and 3.638+/−0.058 ng/mL, respectively. The control rats, F1, M2, M4, and M6, had no measurable simvastatin in their blood plasma at any of the time intervals (FIG. 8).

The average weight of the male rats and female rats was 0.460 kg and 0.291 kg, respectively, and the total amount of blood volume in Long Evans rats is 0.62 mL/kg. The total amount of simvastatin nanotransfersomes diffused into the female rats was 1.59 μg and 2.64 μg for the male rats. The total amount of simvastatin nanotransfersomes in the patch was 20 mg, resulting in a 0.0079% efficiency in the females and 0.01318% efficiency of males.

### DISCUSSION

In the current study, we designed a method to deliver simvastatin, via nanotransfersomes, to the circulatory system of Long Evans rats over a 48-hour time period. Additionally, we observed comparable simvastatin blood plasma concentrations in our study to those reported with oral ingestion of 20 mg/kg of simvastatin in rats⁴⁴. Previously, reported blood plasma concentrations in rats after 4 hours of having ingested 20 mg/kg were found to be 150 ng/mL and increased to 180 ng/mL, while our average concentrations after 6 hours were 34.25 ng/mL and increased to 46.103 ng/mL after 24 hours for females and 25.164 ng/mL at 6 hours and 60.856 ng/mL after 24 hours for males⁴⁴. The difference among males and females shows that the males had a higher average concentration of simvastatin in the blood plasma at the 24-hour period, which could be a result of age differences among the rats. One plausible explanation may be due to CYP3A enzyme function decreasing with age, preventing the metabolism of simvastatin and increasing simvastatin blood plasma concentrations^{45, 46}. The male and female rats used in this study varied between 9-12 months in age. Among the males in this study, differences in simvastatin blood plasma concentration also occurred. Specifically, M3 of the male rats had a simvastatin blood plasma concentration of 26.78 ng/mL, which was significantly lower than the simvastatin blood plasma concentrations of M1 and M5, which were 87.89 ng/mL and 67.905 ng/mL, respectively. This particular male rat, M3, may have had higher CYP3A enzyme function due to age or metabolic state compared to M1 and M5, resulting in the significant decrease of simvastatin blood plasma concentration during the 24-hour time interval; however, CYP enzyme levels were not measured to confirm. The transdermal capabilities of the simvastatin nanotransfersomes, while lower than oral ingestion, allows for a prolonged dosage time at a lower concentration, which eliminates the potential for accidental overdosing^{47, 48}.

The lower diffusion efficiency of the in vivo experiment compared to the skin-PAMPA assay is significant. Previous reports have noted an 88% predictive capability when using a C12 and C18 synthesized certramide³⁰. However, this study comparing the transdermal capabilities of simvastatin nanotransfersomes between an in vitro skin-PAMPA technique and an in vivo transdermal delivery study showed a significant decrease in the predictive capabilities of the skin-PAMPA. The percentage of the diffused drug to the loaded drug of the in vitro skin-PAMPA technique was 20.1% after 6 hours using the skin-PAMPA technique, while only 0.00325% for females and 0.00372% for males at the 6-hour interval for the in vivo study. Additionally, even after the 48-hour interval, the percentage of diffused drug for the in vivo study only accumulated to 0.0079% for females and 0.01318% for males. This significant decrease in diffused simvastatin compared to the total loaded amount of simvastatin loaded could be a result of the gelatin carrier that the liposomes were loaded into. The gelatin chosen was a cold-water fish skin gelatin due to the high gelling temperature, which allowed for a viscous aqueous solvent to be utilized without requiring longer diffusion times through a semi-solid lattice structure²⁶. Another potential reason for the lower percentage of simvastatin diffused is that a thicker dermal membrane of the in vivo study compared to the membrane used in the in vitro study may have limited the total amount of nanotransfersomes detected. This may have occurred due to a longer diffusion distance of the in vivo dermal membrane, which would prevent the diffusion to the circulatory system of the Long Evans rats. The total distance required to cross the Skin-PAMPA is only 0.45 μm, while the skin of a Long Evans rat has a skin thickness of 2.04-2.80 mm⁴⁹.

Future studies of this transdermal patch should investigate a hydrogel as the carrier for the nanotransfersomes. For example, hydrogel consisting of B-Chitosan may be able to overcome the challenge of diffusion through a controlled release of nanotransfersomes⁵⁰. This study has found that a gelatin hydrogel was able to release pulsed doses of liposomes from the gelatin upon heating. Additionally, the gelatin has a low gelling point, but upon heating the semi-solid gel, the structured lattice shrinks in size and expels out the liposomes from the hydrogel, releasing a known dose of liposomes. A dose control mechanism for these transdermal nanotransfersomes should be further studied on this nanotransfersomes and subsequently studied using the skin-PAMPA technique to determine potential dose control possibilities of long-term transdermal patches.

This simvastatin transdermal delivery patch will also aid in the reduction of ischemic heart disease through the added benefit of drug compliance. Currently, ischemic heart disease is the leading cause of death in the world and has been correlated to high levels of total cholesterol in the blood^{51, 52}. Specifically, 50% of all fatal cases of ischemic heart disease have found higher levels of cholesterol in the blood, due to reduced patient compliance, with less than 50% of diagnosed patients complying with their statin treatment after 2 years^{54 53}. This transdermal simvastatin patch developed will aid in patient compliance, as the patient will not have to remember to take their medications daily due to the prolonged dosage over 48 hours.

### REFERENCES

1) S. Korani, et al., Application of nanotechnology to improve the therapeutic benefits of statins. Drug discovery today. 2019; 24:567-574
2) A. Oesterle, U. Laufs and J. K. Liao, Pleiotropic effects of statins on the cardiovascular system. Circulation research. 2017; 120:229-243
3) S. Padhye and M. S. Nagarsenker, Simvastatin solid lipid nanoparticles for oral delivery: formulation development and in vivo evaluation. Indian journal of pharmaceutical sciences. 2013; 75:591
4) Y. Shitara and Y. Sugiyama, Pharmacokinetic and pharmacodynamic alterations of 3-hydroxy-3-methylglutaryl coenzyme A (HMG-CoA) reductase inhibitors: drug-drug interactions and interindividual differences in transporter and metabolic enzyme functions. Pharmacology & therapeutics. 2006; 112:71-105
5) S. Bose, Nano drug delivery system in pharmacy and chemistry review article. IOSR Journal of Pharmacy and Biological Sciences. 2015; 10:1-6
6) J. K. Patra, G. Das, L. F. Fraceto, E. V. R. Campos, M. del Pilar Rodriguez-Torres, L. S. Acosta-Torres, et al., Nano based drug delivery systems: recent developments and future prospects. Journal of nanobiotechnology. 2018; 16:71
7) A. K. Seth, 2019. Transdermal Drug Therapy: Emerging Techniques and Improved Patient Compliance Novel Drug Delivery Technologies, Springer, pp 261-289
8) M. Mezei and V. Gulasekharam, Liposomes—a selective drug delivery system for the topical route of administration I. Lotion dosage form. Life Sciences. 1980; 26:1473-1477
9) G. G. D'Souza, 2016. Liposomes, Springer.
10) M. M. Nounou, L. K. El-Khordagui, N. A. Khalafallah and S. A. Khalil, Liposomal formulation for dermal and transdermal drug delivery: past, present and future. Recent patents on drug delivery & formulation. 2008; 2:9-18
11) M. Amjadi, B. Mostaghaci and M. Sitti, Recent advances in skin penetration enhancers for transdermal gene and drug delivery. Current gene therapy. 2017; 17:139-146
12) T. Badenhorst, D. Svirskis and Z. Wu, Pharmaceutical strategies for the topical dermal delivery of peptides/proteins for cosmetic and therapeutic applications. Austin J. Pharmacol. Ther. 2014; 2:1-10
13) H. R. A. Ashtiani, P. Bishe, N.-A. Lashgari, M. A. Nilforoushzadeh and S. Zare, Liposomes in cosmetics. Journal of Skin and Stem Cell. 2016; 3
14) M. Li, C. Du, N. Guo, Y. Teng, X. Meng, H. Sun, et al., Composition design and medical application of liposomes. European journal of medicinal chemistry. 2019; 164:640-653
15) M. Ashtikar, K. Nagarsekar and A. Fahr, Transdermal delivery from liposomal formulations-Evolution of the technology over the last three decades. Journal of Controlled Release. 2016; 242:126-140
16) S. H. A. El-Alim, A. A. Kassem, M. Basha and A. Salama, Comparative study of liposomes, ethosomes and transfersomes as carriers for enhancing the transdermal delivery of diflunisal: In vitro and in vivo evaluation. International Journal of Pharmaceutics. 2019; 563:293-303
17) A. Gupta, G. Aggarwal, S. Singla and R. Arora, Transfersomes: a novel vesicular carrier for enhanced transdermal delivery of sertraline: development, characterization, and performance evaluation. Scientia pharmaceutica. 2012; 80:1061-1080
18) A. Ahad, et al, Formulation and optimization of nanotransfersomes using experimental design technique for accentuated transdermal delivery of valsartan. Nanomedicine: nanotechnology, biology and medicine. 2012; 8:237-249
19) P. Chaurasiya, et al, Transfersomes: a novel technique for transdermal drug delivery. Journal of Drug Delivery and Therapeutics. 2019; 9:279-285
20) I. W. De Silva, et al, A comparative study of microporous polyolefin silica-based paper and cellulose paper substrates utilizing paper spray-mass spectrometry in drug analysis. Analytical Methods. 2019; 11:3066-3072
21) B. S. Cook, Y. et al., Inkjet catalyst printing and electroless copper deposition for low-cost patterned microwave passive devices on paper. Electronic Materials Letters. 2013; 9:669-676
22) F. Albu, M. D. Toltica and E. F. Petrut, Vulnerabilities of the Romanian ID Card. Eur. J. Pub. Ord. & Nat'l Sec. 2016:19
23) S. D. Hellring, E. A. Furar, R. M. Peffer, M. Sandala, S. Duffy and P. L. Votruba-Drzal, 2019. Method for preparing a resin-treated microporous membrane, Google Patents,
24) R. Clarke, R. et al, 2007. Gas-permeable membrane, Google Patents,
25) A. Gennadios, et al, Application of edible coatings on meats, poultry and seafoods: a review. LWT-Food Science and Technology. 1997; 30:337-350
26) J. Wu, et al., The preparation, characterization, antimicrobial stability and in vitro release evaluation of fish gelatin films incorporated with cinnamon essential oil nanoliposomes. Food Hydrocolloids. 2015; 43:427-435
27) S. Young, et al, Gelatin as a delivery vehicle for the controlled release of bioactive molecules. Journal of controlled release. 2005; 109:256-274
28) Z. M. Barzoki, Z. Emam-Djomeh, E. Mortazavian, N. Rafiee-Tehrani, H. Behmadi, M. Rafiee-Tehrani, et al., Determination of diffusion coefficient for released nanoparticles from developed gelatin/chitosan bilayered buccal films. International journal of biological macromolecules. 2018; 112:1005-1013
29) Y. Mao, X. Li, G. Chen and S. Wang, Thermosensitive hydrogel system with paclitaxel liposomes used in localized drug delivery system for in situ treatment of tumor: better antitumor efficacy and lower toxicity. Journal of pharmaceutical sciences. 2016; 105:194-204
30) B. Sinkó, et al., Skin-PAMPA: A new method for fast prediction of skin penetration. European Journal of Pharmaceutical Sciences. 2012; 45:698-707
31) B. Sinkó, G. Vizserálek and K. Takács-Novák, Skin PAMPA: Application in practice. ADMET and DMPK. 2014; 2:191-198
32) B. Sinkó, J. Kökösi, A. Avdeef and K. Takács-Novák, A PAMPA study of the permeability-enhancing effect of new ceramide analogues. Chemistry & biodiversity. 2009; 6:1867-1874
33) K. Clemons, A. Kretsch and G. Verbeck, Parallel artificial membrane permeability assay for blood-brain permeability determination of illicit drugs and synthetic analogues. Sci Justice. 2014; 54:351-5
34) Y. Kubo, E. Fukui, S.-I. Akanuma, M. Tachikawa and K.-I. Hosoya, Application of membrane permeability evaluated in in vitro analyses to estimate blood-retinal barrier permeability. Journal of pharmaceutical sciences. 2012; 101:2596-2605
35) M. Chioua, et al., α-Aryl-N-alkyl nitrones, as potential agents for stroke treatment: synthesis, theoretical calculations, antioxidant, anti-inflammatory, neuroprotective, and brain-blood Barrier permeability properties. Journal of medicinal chemistry. 2011; 55:153-168
36) J. Mensch, A. et al, Evaluation of various PAMPA models to identify the most discriminating method for the prediction of BBB permeability. European journal of pharmaceutics and biopharmaceutics. 2010; 74:495-502
37) D. Jhala, et al, Optimization and validation of an in vitro blood brain barrier permeability assay using artificial lipid membrane. J. Bioequiv. Availab. 2012; 14:1-5
38) T. J. Franz, Percutaneous absorption. On the relevance of in vitro data. Journal of Investigative Dermatology. 1975; 64:190-195
39) T. J. Franz, 1978. The finite dose technique as a valid in vitro model for the study of percutaneous absorption in man Skin-Drug Application and Evaluation of Environmental Hazards, Karger Publishers, pp 58-68
40) S. Hua, Lipid-based nano-delivery systems for skin delivery of drugs and bioactives. Frontiers in pharmacology. 2015; 6:219
41) T. Kanno, T. Yamada, H. Iwabuki, H. Tanaka, S. i. Kuroda, K. Tanizawa, et al., Size distribution measurement of vesicles by atomic force microscopy. Analytical biochemistry. 2002; 309:196-199
42) B. Sinkó, et al, Synthesis and characterization of long-chain tartaric acid diamides as novel ceramide-like compounds. Molecules. 2010; 15:824-833
43) A. Avdeef, 2012. Absorption and drug development: solubility, permeability, and charge state, John Wiley & Sons.
44) D. Xu, F. Li, M. Zhang, J. Zhang, C. Liu, M.-y. Hu, et al., Decreased exposure of simvastatin and simvastatin acid in a rat model of type 2 diabetes. Acta Pharmacologica 10 Sinica. 2014; 35:1215
45) M. H. Davidson, Does differing metabolism by cytochrome P450 have clinical importance? Current atherosclerosis reports. 2000; 2:14-19
46) S.-F. Xu, et al, Age-associated changes of cytochrome P450 and related phase-2 gene/proteins in livers of rats. PeerJ. 2019; 7:e7429
47) M. S. Khan and M. S. Roberts, Challenges and innovations of drug delivery in older age. Advanced drug delivery reviews. 2018; 135:3-38
48) S. Tyagi and K. Goyal, Transdermal drug delivery system: Quality approaches and evaluation. Innovat International Journal Of Medical & Pharmaceutical Sciences. 2017; 2
49) R. L. Bronaugh, R. F. Stewart and E. R. Congdon, Differences in permeability of rat skin related to sex and body site. J. Soc. Cosmet. Chem. 1983; 34:135
50) H. S. O'Neill, C. C. Herron, C. L. Hastings, R. Deckers, A. L. Noriega, H. M. Kelly, et al., A stimuli responsive liposome loaded hydrogel provides flexible on-demand release of therapeutic agents. Acta biomaterialia. 2017; 48:110-119
51) A. Varbo, M. Benn, A. Tybjorg-Hansen, A. B. Jorgensen, R. Frikke-Schmidt and B. G. Nordestgaard, Remnant cholesterol as a causal risk factor for ischemic heart disease. Journal of the American College of Cardiology. 2013; 61:427-436
52) D. Kwon, et al, Total cholesterol and mortality from ischemic heart disease and overall cardiovascular disease in Korean adults. Medicine. 2019; 98
53) S. Patani, et al, Ischemic heart disease: role of total cholesterol: HDLC ratio as an important indicator compared to LDLC. Int J Biochem Biotechnol. 2018; 14:13-7
54) C. A. Jackevicius, M. Mamdani and J. V. Tu, Adherence with statin therapy in elderly patients with and without acute coronary syndromes. Jama. 2002; 288:462-467

A number of embodiments of the invention have been described. Nevertheless, it can be understood that various modifications may be made without departing from the spirit and scope of the invention. Accordingly, other embodiments are within the scope of the following claims.

1: A product of manufacture for the transdermal or transmucosal delivery of compounds, comprising at least three layers:
(a) an inner layer designed to be approximate to a mucous membrane or skin comprising a heat sensitive porous material, wherein the heat sensitive porous material becomes porous, semi-solid or soluble at a temperature of between about 50° C. to 68° C., or at about 60° C., or the heat sensitive porous material comprises a plurality of pores which expand in pore size or pore diameter at a temperature of between about 50° C. to 68° C., or at about 60° C., or the viscosity of the heat sensitive porous material increases at a temperature of between about 50° C. to 68° C., or at about 60° C.
(b) an inner or middle layer comprising a plurality of nanotransferosomes, lipid bodies, nano-liposomes, liposomes, nano-liposomes or equivalent particles comprising or having contained therein a payload or an active agent, and
(c) an outer layer comprising or substantially comprising a Positive Temperature Coefficient (PTC) material.

2: The product of manufacture of claim 1, further comprises a fourth layer comprising a micro-porous or a mesoporous substrate comprising an embedded, or having contained therein, a gas, a gel or a liquid which expands when heated to between about 50° C. to 68° C.,
wherein optionally the embedded or contained gas, gel or liquid expands in volume by about 10% to 100%,

3: The product of manufacture of claim 1, wherein the product of manufacture is fabricated on or as a wearable product or a device, wherein the wearable product or device is or comprises a band, an eyeglass, a watch, clothes, shoes, a hat, a prosthesis, a wound or burn dressing, a patch, or any garment or wearable product.

4: The product of manufacture of claim 3, wherein the wearable product or device comprises an adhesive such that the product of wearable product or device can be removably attached or adhered to the skin or a mucous membrane.

5: The product of manufacture of any of the preceding claims, further comprising a heating module capable of heating the product of manufacture, the fourth layer comprising a nanoporous, micro-porous or a mesoporous substrate, and/or the heat sensitive porous material of the inner later to a temperature of between about 55° C. to about 65° C., or to about 60° C.,
wherein optionally the heating module comprises a switch which when manually or electronically activated turns the heating module on (to produce heat) or off.

6: The product of manufacture of claim 4, wherein the product of manufacture further comprises an antenna (optionally an ultra-thin magnetic spiral antenna) or equivalent, and a near-field communication (NFC) chip or equivalent, and the ultra-thin magnetic spiral antenna is capable of receiving a remote electromagnetic signal and transmitting the signal to the near-field communication (NFC) chip, and the NFC chip is operatively connected to the heating module.

7: A method of transdermally or transmucosally administering a payload or an active agent to an individual in need thereof, comprising:
(a) providing a product of manufacture of any of the preceding claims, wherein the nanotransferosomes, lipid bodies, nano-liposomes, liposomes, nano-liposomes or equivalent particle comprise or have contained therein the payload or active agent,
(b) applying, contacting or placing in close approximation the inner surface of the product of manufacture to a skin or a mucous membrane; and
(c) (i) applying sufficient heat for a sufficient time to the heat sensitive porous material to melt or sufficiently solubilize the heat sensitive porous material to allow passage of a plurality of nanotransferosomes, lipid bodies, nano-liposomes, liposomes, nano-liposomes or equivalent particles from (or out of) the product of manufacture to the surface of the skin or the mucous membrane, or
(ii) generating a remote signal that is transmitted to the antenna and the near-field communication (NFC) chip to generate a sufficient signal to activate the heating module for a sufficient time to the heat sensitive porous material to melt or sufficiently solubilize the heat sensitive porous material to allow passage of a plurality of liposomes or nano-liposomes from (or out of) the product of manufacture to the surface of the skin or the mucous membrane,
and optionally sufficient heat for a sufficient time is applied to the heat sensitive porous material such that all, substantially all or only a portion of the active agent-comprising nanotransferosomes, lipid bodies, nano-liposomes, liposomes, nano-liposomes or equivalent particles pass from (or out of) the product of manufacture to the surface of the skin or the mucous membrane.

8: The method of claim 7, wherein two, three or multiple pulses of sufficient heat for a sufficient time are applied to the product of manufacture such that only portions of the active agent-comprising nanotransferosomes, lipid bodies, nano-liposomes, liposomes, nano-liposomes or equivalent particles pass from or out of the product of manufacture to the surface of the skin or the mucous membrane pass from the product of manufacture to the skin or mucous membrane,
and optionally the amount of payload- or active agent-comprising nanotransferosomes, lipid bodies, nano-liposomes, liposomes, nano-liposomes or equivalent particles that pass from (or out of) the product of manufacture to the surface of the skin or the mucous membrane is controlled by the amount of heat generating by the heating module or the amount of time the heating module is heated,
and optionally for each pulse heat between about 5% to about 95%, or 10% to about 90%, or about 20% to 80%, of the payload- or active agent-comprising nanotransferosomes, lipid bodies, nano-liposomes, liposomes, nano-liposomes or equivalent particles pass from the product of manufacture to the skin or mucous membrane.

9-10. (canceled)

11: A kit comprising a product of manufacture of claim 1.

12: The product of manufacture of claim 1, wherein in step (a) the heat sensitive porous material becomes porous, semi-solid or soluble at a temperature of between about 55° C. to 65° C.

13: The product of manufacture of claim 1, wherein in step (a) the heat sensitive porous material comprises a plurality of pores which expand in pore size or pore diameter at a temperature of between about 55° C. to 65° C.

14: The product of manufacture of claim 1, wherein in step (a) the viscosity of the heat sensitive porous material increases at a temperature of between about 55° C. to 65° C.

15: The product of manufacture of claim 1, wherein the heat sensitive porous material comprises a gelatin film or a hydrogel, or equivalents, and optionally the gelatin film comprises a fish gelatin or equivalent, and optionally the fish gelatin comprises a fish skin gelatin, a bovine gelatin, a porcine gelatin or a hydrogel or equivalents.

16: The product of manufacture of claim 1, wherein the heat-sensitive porous material or equivalent is layered on or embedded in a microporous polyolefin silica substrate or equivalent.

17: The product of manufacture of claim 1, wherein the average pore size or pore diameter increases to between about 50 nm to about 50 μm when the temperature of the heat sensitive porous material increases to between about 30° C. to about 68° C.

18: The product of manufacture of claim 1, wherein the viscosity of the heat sensitive porous material increases by between about 5% to about 100% when the temperature of the heat sensitive porous material increases to between about 50° C. to 68° C.

19: The product of manufacture of claim 1, wherein the payload or active agent comprises a small molecule, a protein or peptide, a polysaccharide, a lipid, an ion, a reagent, a pharmaceutical or drug, an electrolyte, a natural product and/or a nutraceutical.

20: The product of manufacture of claim 1, wherein all or substantially all of the plurality of payload-comprising nanotransferosomes, lipid bodies, nano-liposomes, liposomes, nano-liposomes or equivalent particle have a diameter of less than about 30 nm, or at or less than about 25 nm.

21: The product of manufacture of claim 1, wherein all or substantially all of the plurality of payload-comprising nanotransferosomes, lipid bodies, nano-liposomes, liposomes, nano-liposomes or equivalent particle have a diameter averaging between about 20 and 30 nm, or between about 20 and 25 nm.

22: The product of manufacture of claim 1, wherein:
the plurality of payload-comprising nanotransferosomes, lipid bodies, nano-liposomes, liposomes, nano-liposomes or equivalent particle are embedded in a microporous, mesoporous or nanoporous polyolefin silica substrate, or equivalent, or are embedded in an organic or inorganic porous framework,
the PTC material comprises a poly-crystalline ceramic or silica material, or a microporous polyolefin silica substrate,
the poly-crystalline ceramic or silica material, or the microporous polyolefin silica substrate, comprises pores, wherein all or substantially all of the pores are less than about 30 nm in diameter, or less than about 25 nm, or average less than about 30 nm in diameter,
the poly-crystalline ceramic material or the microporous polyolefin silica substrate comprises a dopant to make the poly-crystalline ceramic material or the microporous polyolefin silica substrate conductive,
the PTC material is printed onto, attached to or embedded on or contained within a polymer, a textile, a fabric or a cloth, and/or
the polymer comprises a polyester, a polyethylene terephthalate (PET), an acrylic-comprising polymer or equivalent.
